(19) United States
(12) Patent Application Publication
(10) Pub. No.: US 2022/0275320 A1
(43) Pub. Date: Sep. 1, 2022

(54) MICROALGAE INCUBATOR FOR PRODUCTION OF BIOFUEL

(71) Applicant: Jae Hoon Kim, Seoul (KR)

(72) Inventor: Jae Hoon Kim, Seoul (KR)

(21) Appl. No.: 17/359,763

(22) Filed: Jun. 28, 2021

(30) Foreign Application Priority Data
Feb. 26, 2021 (KR) 10-2021-0026781

(51) Int. Cl. C12M 1/00; C12M 1/34; A01G 33/00; C12N 1/12

(52) U.S. Cl. CPC C12M 41/14; C12M 41/34; C12M 23/02; A01G 33/00; C12N 1/12

(57) ABSTRACT

Disclosed herein is a microalgae incubator. The microalgae incubator is formed as a container in the form of a barrel having an open upper surface and a predetermined depth to accommodate water containing microalgae. The microalgae incubator has a regular polygonal cross section having an even number of vertices or a circular cross section. The microalgae incubator includes an air pipe configured such that an inner tube is located inside the microalgae incubator along a direction passing through a center of a cross section in a lower portion of a body of the microalgae incubator and both ends of the inner tube form connection holes in a surface of the body of the microalgae incubator.

### CROSS-REFERENCE TO RELATED APPLICATION

This application claims the benefit of Korean Patent Application No. 10-2021-0026781 filed on Feb. 26, 2021, which is hereby incorporated by reference herein in its entirety.

### BACKGROUND

### 1. Technical Field

The present invention relates to a microalgae incubator capable of efficiently culturing microalgae in a large-scale facility for the production of biofuel.

### 2. Description of the Related Art

Advances across the scientific, medicinal and industrial fields have led to rapid population growth. As the population increases, energy consumption continues to increase. As the use of fossil fuels, which are the basis of energy that supports society today, is rapidly increasing, the imbalance in the supply and demand of energy becomes serious internationally. Since fossil fuels are buried intensively in specific areas, the imbalance in the supply and demand of energy can lead to political and diplomatic conflicts and disputes.

Therefore, interest and efforts in the development of alternative energy are increasing around the world in order to overcome various problems such as the regional concentration of fossil fuels, the depletion of fossil fuels, and increases in the cost of collection of fossil fuels. As a branch, a technology for producing alternative fuels from natural resources that can be obtained in large quantities through cultivation, i.e., a technology for producing biofuel, is attracting attention. Biofuel is a fuel obtained from biomass, and includes not only living organisms but also by-products from metabolic activities such as animal excrement. Biofuel is renewable energy different from fossil fuels, and includes bioethanol and biodiesel.

There are many types of biomass that produce biofuels. Among them, the promising biomass is microalgae. Microalgae are a collective term for a group of organisms having a considerably small size in the form of single cells that undergo photosynthesis, and have the advantage of being able to grow more efficiently and faster than terrestrial plants. In addition, microalgae have a great advantage in that it is a natural raw material that can minimize the impact on the world's food supply and demand. In other words, biofuels have been mainly produced from corn, soybeans, and sugarcane. When such crops become the main raw materials of biofuels, food shortages may occur due to increased food prices due to fewer grains for people to eat. In contrast, microalgae are biomass that does not cause such concerns.

However, in order for microalgae to be economical as biomass, it is a problem necessary to be solved that it must be cultivated on a large scale. In other words, a facility for cultivating microalgae must be prepared on a large scale of several hundred hectares in order to be competitive with fossil fuels. Furthermore, there is a need to provide a means to effectively harvest microalgae in such a large-scale culture facility.

Accordingly, the present applicant has invented a type of bio-plant apparatus 10 for culturing and harvesting microalgae on a large scale, as shown in FIG. 1. The apparatus of FIG. 1 is an automated harvesting apparatus that collects microalgae from microalgae incubators 1000, connected vertically and horizontally on a large scale, by using a gantry crane 100.

However, the considerably high initial installation cost is incurred by a facility that installs microalgae incubators on a large scale and harvests microalgae through an automated process therefrom. There is required the work of aligning the microalgae incubators that are connected over more than several thousand kilometers in the lengthwise direction, and the work of installing an air pipe to circulate microalgae and supply CO2 to each microalgae incubator also requires a lot of labor. In addition, there is a need to effectively reduce the cost of transporting thousands to tens of thousands of microalgae incubators on a large scale.

### RELATED ART DOCUMENT

Patent document: Korean Patent Application Publication No. 10-2020-0108745 (published on Sep. 21, 2020)

### SUMMARY

An object of the present invention is to provide a microalgae incubator suitable for the efficient construction of a large-scale microalgae culture facility.

According to an aspect of the present invention, there is provided a microalgae incubator formed as a container in the form of a barrel having an open upper surface and a predetermined depth to accommodate water containing microalgae, the microalgae incubator having a regular polygonal cross section having an even number of vertices or a circular cross section, the microalgae incubator including an air pipe configured such that an inner tube is located inside the microalgae incubator along a direction passing through a center of a cross section in a lower portion of a body of the microalgae incubator and both ends of the inner tube form connection holes in a surface of the body of the microalgae incubator.

The air tube and the body of the microalgae incubator may be formed in an integrated manner.

One or more air holes opened upward may be formed in the central region of the inner tube.

Each of the connection holes may be configured as a one-touch nipple.

A pair of hook and cutout may be formed on the edge of the upper end of the body of the microalgae incubator in a direction perpendicular to a direction in which the air tube is extended; and the hook may be fixed in the cutout by being fitted into the cutout.

The body of the microalgae incubator may be shaped in the form of a truncated cone with a wider upper portion.

### BRIEF DESCRIPTION OF THE DRAWINGS

The above and other objects, features, and advantages of the present invention will be more clearly understood from the following detailed description taken in conjunction with the accompanying drawings, in which:

FIG. 1A and FIG. 1B are views showing an example of a bio-plant apparatus for culturing and harvesting microalgae on a large scale;

FIG. 2A and FIG. 2B are perspective views showing a microalgae incubator according to the present invention;

FIG. 3 is a view showing a state in which the microalgae incubators of FIG. 2A and FIG. 2B are arranged and connected in the lengthwise direction;

FIG. 4 is a view showing a state in which the microalgae incubators of FIG. 2A and FIG. 2B are arranged and connected in the widthwise direction; and

FIG. 5 is a view showing a state in which a plurality of microalgae incubators is superimposed on top of each other.

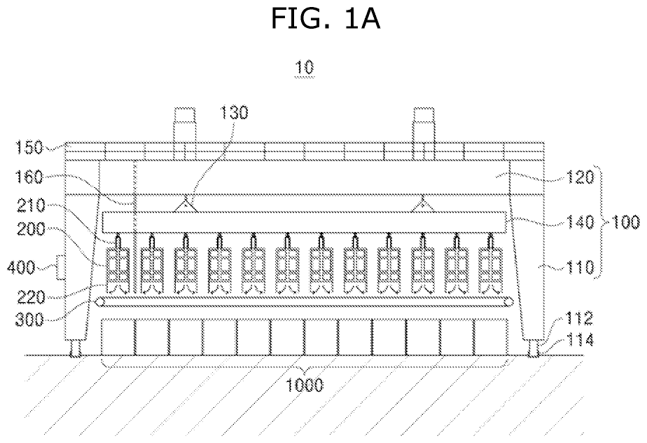

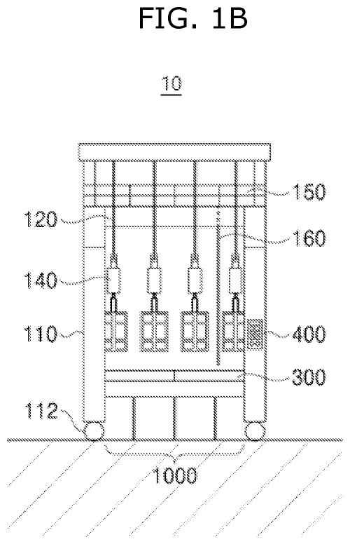

### DETAILED DESCRIPTION

Embodiments of the present invention will be described in detail below with reference to the accompanying drawings. The advantages and features of the present invention and methods for achieving them will be apparent with reference to the embodiments described below in detail in conjunction with the accompanying drawings. However, the present invention is not limited to the embodiments to be described below, but may be implemented in various different forms. The embodiments are provided merely to make the disclosure of the present invention complete, and to fully convey the scope of the invention to those of ordinary skill in the art. The invention is defined only based on the scope of the claims. The same reference symbols refer to the same components throughout the specification.

Unless otherwise defined, all terms (including technical and scientific terms) used herein may be used in senses that can be commonly understood by those of ordinary skill in the art to which the present invention pertains. In addition, the terms defined in commonly used dictionaries are not interpreted ideally or excessively unless explicitly defined specifically. The terms used herein are intended to describe embodiments, but are not intended to limit the present invention. In this specification, a singular expression also includes a plural expression unless specifically stated in the phrase.

As used herein, “comprises” and/or “comprising” means that recited components, steps, operations and/or elements do not exclude the presence or addition of components, steps, operations, and/or elements.

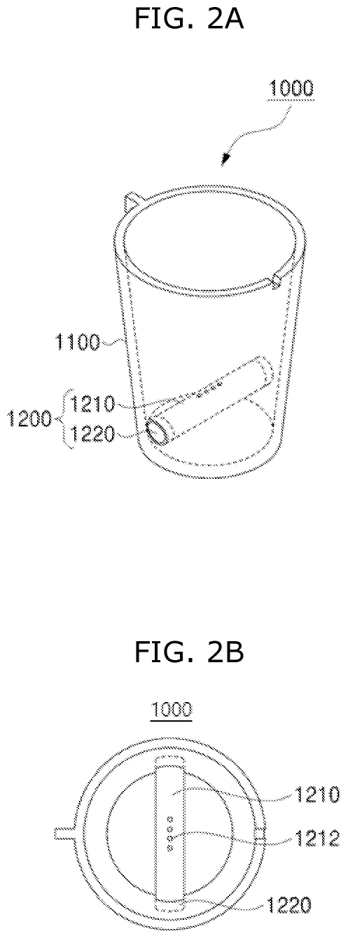

FIG. 2A and FIG. 2B are perspective views showing a microalgae incubator 1000 according to the present invention.

Referring to FIG. 2A and FIG. 2B, the microalgae incubator 1000 is configured as a container in the form of a barrel having an open upper surface and a predetermined depth to accommodate water containing microalgae. In this case, the cross-sectional shape of the microalgae incubator 1000 according to the present invention preferably forms a regular polygonal cross section having vertical, horizontal and vertical symmetry and an even number of vertices, or preferably forms a circular cross section. The reason for this is that the cross section is suitable for the symmetrical arrangement of an air tube 1200 and a latch 1300 and a cutout 1310 perpendicular to the air tube 1200, which will be described later. The microalgae incubator 1000 is shown as having a circular cross-section in the drawing as an example.

The air tube 1200 disposed along the direction passing through the center of the cross section is provided in the lower portion of the body 1100 of the microalgae incubator 1000. The air tube 1200 is intended to introduce air containing CO2 so that the microalgae contained in the microalgae incubator 1000 can perform photosynthesis. Furthermore, it is preferable to produce the microalgae incubator 1000 with a material and color that can transmit sunlight therethrough desirably so that photosynthesis occurs in the microalgae contained therein.

The air pipe 1200 includes an inner tube 1210 that is located inside the microalgae incubator 1000 along a direction passing through the center of the cross-section in the lower portion of the body 1100 of the microalgae incubator 1000. Both ends of the inner tube 1210 form connection holes 1220 in the surface of the body 1100 of the microalgae incubator 1000. In other words, the inner tube 1210 may be viewed as a tube member that passes through the lower portion of the body 1100 of the microalgae incubator 1000. Both ends of the inner tube 1210 rarely protrude from the surface of the body 1100. Since the air tube 1200 passes through the center of the cross section, outer tubes are disposed completely opposite each other in the radial direction. However, the lengths by which the individual outer tubes forming the pair of outer tubes protrude may be different.

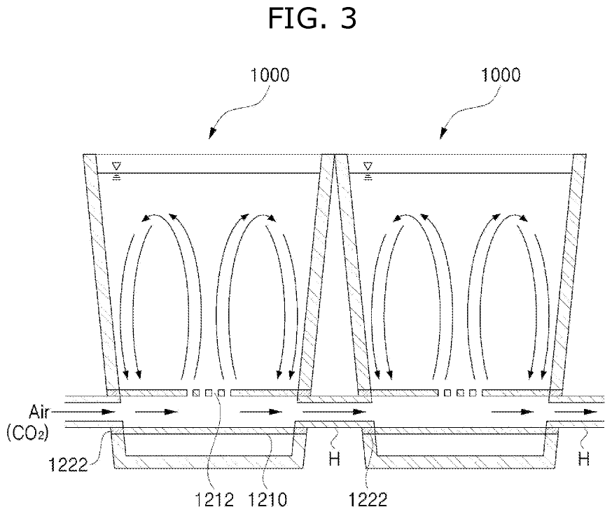

FIG. 3 is a view showing a state in which microalgae incubators 1000 of the present invention are arranged and connected in the lengthwise direction. Referring to FIG. 3, the right (rear) connection hole 1220 of the microalgae incubator 1000 on the left side communicates with the left (front) connection hole 1220 of the microalgae incubator 1000 on the right side through a hollow connection member such as a hose H or a pipe. As such, the inner tubes 1210 of respective microalgae incubators 1000 form a continuous air supply pipe by connecting the connection holes 1220 of the microalgae incubators 1000, adjacent to one another in the longitudinal direction, with hollow connection members.

A plurality of microalgae incubators 1000 may be arranged consecutively such that the inner tubes 1210 are connected to each other in the above-described manner. When pressurized air is supplied to the connection hole 1220 of the outer microalgae incubator 1000, air is supplied to all the microalgae incubators 1000 connected in a line at once.

As described above, in the case of the microalgae incubators 1000 of the present invention, an air supply conduit in the longitudinal direction is extended using the inner pipes 1210 thereof, and thus they have the advantage of reducing installation time and cost compared to the case of simply arranging the microalgae incubators 1000 and installing separate pipes one by one. In particular, in the microalgae incubator 1000 of the present invention, the air tube 1200 and the body 1100 of the microalgae incubator 1000 are manufactured in an integrated manner, e.g., they are manufactured by extrusion molding in an integrated manner, thereby significantly reducing piping cost

In this case, according to an embodiment of the present invention, the connection hole 1220 of the air tube 1200 may be configured as a one-touch nipple 1222. The one-touch nipple 1222 refers to a type of nipple that allows connection to be completed without additional tightening simply by inserting the hose H into a hole. When the connection hole 1220 is configured as the one-touch nipple 1222, the work of connecting the inner tubes 1210 to each other becomes simpler.

Furthermore, referring to FIG. 2A, FIG. 2B and FIG. 3, air holes 1212 opened upward are formed in the central region of the inner tube 1210. The air holes 1212 formed in the central region of the inner tube 1210 eject air from the lower portion of the microalgae incubator 1000 upward when pressurized air is supplied to the air tube 1200. Accordingly, there is formed a circulation in which the water contained in the microalgae incubator 1000 is raised up from the center by the force of the compressed air and is then lowered to the edge. This circulation of the water makes the microalgae contained in the water continue to move and mix without being stagnant, so that all the microalgae grow evenly.

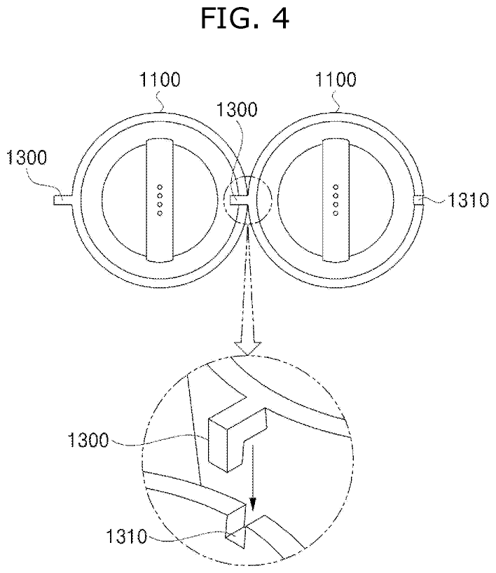

FIG. 4 is a view showing a state in which the microalgae incubators of the present invention are arranged and connected in the widthwise direction. According to an embodiment of the present invention, a coupling structure for alignment in the widthwise direction is provided on the edge of the upper end of the body 1100 of the microalgae incubator 1000. In other words, a pair of hook 1300 and cutout 1310 are formed on the edge of the upper end of the body 1100 of the microalgae incubator 1000 in a direction perpendicular to the direction in which the air tube 1200 is extended. The hook 1300 is fixed in the cutout 1310 by being fitted into the cutout 13.

Accordingly, as shown in FIG. 4, the microalgae incubators 1000 adjacent each other in a lateral direction may be aligned in a lateral direction in such a manner that the microalgae incubators 1000 are arranged in the same direction, i.e., they are arranged such that the cutout 1310 on the left and the hook 1300 on the right face each other, and then the cutout 1310 and the hook 1300 are fitted into each other.

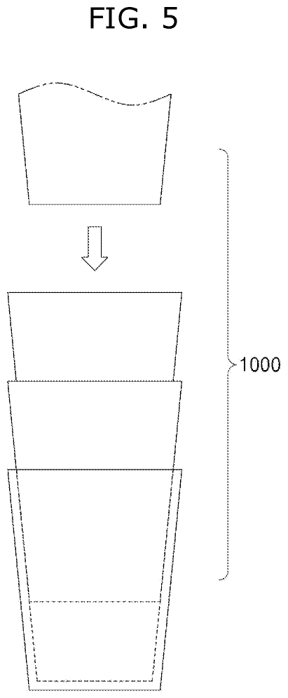

In addition, FIG. 5 is a view showing a state in which a plurality of microalgae incubators 1000 is superimposed on top of each other. As shown in the drawing, the body 1100 of the microalgae incubator 1000 is shaped in the form of a truncated cone with a wider upper portion. Accordingly, the plurality of microalgae incubators 1000 without contents may be superimposed on top of each other as needed, so that the volume thereof can be significantly reduced during storage and transportation, which is a great help in reducing costs.

In the case of the microalgae incubator of the present invention having the above-described configuration, the air tube and the body are formed in an integrated manner, and the microalgae incubators connected in the lengthwise direction provide an air supply structure formed by the connection of the inner tubes of the air tubes. Accordingly, parts where separate air tubes are to be installed are minimized, and thus it may be possible to significantly reduce the installation cost of microalgae incubators installed on a large scale.

Furthermore, the microalgae incubator of the present invention may be provided with the structure by which adjacent microalgae incubators are connected by fitting the hook and the cutout to each other in the widthwise direction, which helps to align the microalgae incubators in the widthwise direction.

In addition, in the present invention, the body of the microalgae incubator is shaped in the form of a truncated cone with a wider upper part. This truncated cone-shaped body allows multiple microalgae incubators to be superimposed and stacked on top of each other, thereby significantly reducing the volume thereof during storage and transportation. This makes a significant contribution to a reduction in cost.

While the embodiments of the present invention have been described above with reference to the accompanying drawings, it will be appreciated by those of ordinary skill in the art to which the present invention pertains that the present invention may be implemented in other specific forms without changing the technical spirit or essential features of the present invention. Therefore, it should be understood that the embodiments described above are illustrative and non-limiting in all respects.

What is claimed is:

1. A microalgae incubator formed as a container in a form of a barrel having an open upper surface and a predetermined depth to accommodate water containing microalgae, the microalgae incubator having a regular polygonal cross section having an even number of vertices or a circular cross section, the microalgae incubator comprising an air pipe configured such that an inner tube is located inside the microalgae incubator along a direction passing through a center of a cross section in a lower portion of a body of the microalgae incubator and both ends of the inner tube form connection holes in a surface of the body of the microalgae incubator.

2. The microalgae incubator of claim 1, wherein the air tube and the body of the microalgae incubator are formed in an integrated manner.

3. The microalgae incubator of claim 1, wherein one or more air holes opened upward are formed in a central region of the inner tube.

4. The microalgae incubator of claim 1, wherein each of the connection holes is configured as a one-touch nipple.

5. The microalgae incubator of claim 1, wherein:
a pair of hook and cutout are formed on an edge of an upper end of the body of the microalgae incubator in a direction perpendicular to a direction in which the air tube is extended; and
the hook is fixed in the cutout by being fitted into the cutout.

6. The microalgae incubator of claim 1, wherein the body of the microalgae incubator is shaped in a form of a truncated cone with a wider upper portion.
